(19) United States
(12) Patent Application Publication
(10) Pub. No.: US 2015/0119388 A1
(43) Pub. Date: Apr. 30, 2015

(54) NOVEL PRO- AND CODRUG DERIVATIVES FOR NANOPARTICLE DELIVERY OF SELECT ANTICANCER AGENTS FORMED USING RAPIDLY CLEAVABLE PHENOLIC ESTER BRIDGES

(71) Applicant: THE CHILDREN'S HOSPITAL OF PHILADELPHIA, PHILADELPHIA, PA (US)

(72) Inventors: Ivan Alferiev, Clemonton, NJ (US); Michael Chorny, Huntingdon Valley, PA (US); Garrett M. Brodeur, Wynnewood, PA (US); Robert J. Levy, Merion Station, PA (US)

(73) Assignee: THE CHILDREN'S HOSPITAL PHILADELPHIA, PHILADELPHIA, PA (US)

(21) Appl. No.: 14/408,064

(22) PCT Filed: Jun. 14, 2013
(86) PCT No.: PCT/US13/45772

§ 371 Date: Dec. 15, 2014

Related U.S. Application Data
(60) Provisional application No. 61/660,219, filed on Jun. 15, 2012.

(51) Int. Cl. A61K 31/5377; A61K 31/365; A61K 9/10; A61K 31/4745

(52) U.S. Cl. CPC A61K 31/5377; A61K 31/4745; A61K 31/365; A61K 9/10
USPC 514/232.8; 546/48; 544/115; 549/298; 514/283; 514/452

(57) ABSTRACT

An ester of ArOH according to the formula R—X—CO—OAr, wherein ArOH is a pharmaceutically active compound selected from the group consisting of SN-38, PI-103, etoposide and fenretinide, wherein a) R is a residue of cholesterol, sitosterol, SN-38, PI-103, etoposide or fenretinide and X is O—CO-L, wherein L is either a direct bond or a linking group including a branched or unbranched hydrocarbyl moiety that may optionally include in-chain or pendant heteroatom substituents and/or cyclic moieties; b) R—X—CO-0 is an all-trans retinoate radical or the 9-cis or 13-cis isomer thereof; or c) R—X— is a branched or unbranched, saturated or unsaturated hydrocarbyl moiety comprising at least 5 carbon atoms and optionally including at least one in-chain or pendant heteroatom substituent and/or cyclic moiety. A dispersion of nanoparticles in an aqueous medium includes nanoparticles including an ester of ArOH according to the formula R—X—CO—OAr wherein ArOH is a pharmaceutically active compound in which Ar is a substituted or unsubstituted aryl or heteroaryl radical, and wherein R is as defined above or R—X—CO-0 is as defined above. The ester or dispersion may be used to treat a diagnosed medical condition in a patient.

### BACKGROUND OF THE INVENTION

Camptothecin and its analogs exhibit potent anticancer activity via interacting specifically with topoisomerase I, an enzyme that relieves torsional strain in DNA by inducing reversible single-strand breaks. Camptothecin and its analogs bind to the topoisomerase I-DNA complex and prevent re-ligation of these single-strand breaks. While showing high potency against various types of malignancies, including colorectal, lung, gastric, cervical and ovarian cancers, malignant lymphoma, glioblastoma and neuroblastoma, camptothecin drugs have a non-specific mode of action, affecting all rapidly dividing cells in the body exposed to the drug. Because in its free form the drug distributes both to the tumor and to healthy non-target cells, the lack of tissue selectivity results in severe toxic effects, including suppression of the immune system and diarrhea. In addition, camptothecin is poorly water soluble, and at physiologic pH undergoes is conversion to the inactive carboxylate form of the drug, which is stabilized by its avid binding to human serum albumin in circulation.

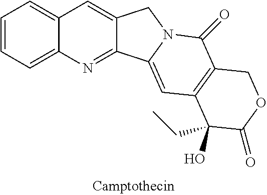 [SMILES: CC[C@@]1(O)C(=O)OCc2c1cc1n(c2=O)Cc2cc3ccccc3nc2-1]

The solubility issues and extensive albumin binding have been partially addressed by designing a dipiperidino derivative, irinotecan, which acts as a water-soluble precursor of a biologically active but poorly soluble camptothecin analog, 7-ethyl-10-hydroxy-20(S)-camptothecin (SN-38).

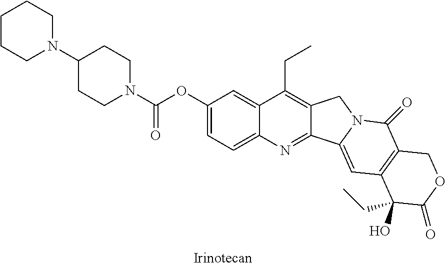 [SMILES: CCc1c2c(nc3ccc(OC(=O)N4CCC(N5CCCCC5)CC4)cc13)-c1cc3c(c(=O)n1C2)COC(=O)[C@]3(O)CC]

However the use of this water-soluble precursor did not address the toxicity issues, as biodistribution still occurs in a non-specific manner and the drug therefore affects healthy cells and tissues. In addition, the rate of the carboxylesterase-mediated conversion of irinotecan to SN-38 is generally less than 10%, i.e., less than 10% of it is converted to the pharmacologically active SN-38, while the rest is eliminated through the alternative pathways. The rate is also affected by the genetic interindividual variability of carboxylesterase activity.

An alternative approach for creating a formulation of SN-38 that would both be injectable in an aqueous non-toxic vehicle and exhibit improved tumor specificity is to form nanoparticles that can take advantage of the Enhanced Permeability and Retention (EPR) effect. The EPR effect enables preferential tumor distribution of constructs in a certain size range. One attempt to do this has been previously reported, and uses a conjugate of SN-38 with a poly(ethylene glycol)-poly(glutamate) block copolymer. The material was tested as a treatment for triple-negative breast cancer and relapsed small cell lung cancer. This amphiphilic conjugate, which has SN-38 covalently bound to the poly(glutamate) segment by the condensation reaction between the carboxylic acid on the polymer and the phenol hydroxyl on SN-38, self assembles in water into polymeric micelles with a size compatible with EPR. Although this conjugate was more effective in achieving high local concentrations of SN-38 in tumor tissue and inhibiting tumor growth compared to irinotecan in recent animal studies, its maximal tolerated dose was found to be considerably lower than that of irinotecan, indicating that adverse effects may still remain a major limiting factor to its clinical utility.

Adverse reactions due to lack of tissue specificity and issues with solubility and chemical stability, similar to those seen with camptothecin drugs, are commonly seen in other pharmacological and chemical families as well and pose limitations to their effective use as anticancer agents. One example is etoposide, a semisynthetic podophyllotoxin derivative acting as a topoisomerase II inhibitor. Similar to camptothecin drugs, it is also effective against a broad range of tumors, both adult and pediatric, but its therapeutic use is limited by poor water solubility and adverse effects, mainly myelosuppression). Thus, improved methods of delivering these and other anticancer agents are needed.

### SUMMARY OF THE INVENTION

In one aspect, the invention provides an ester of ArOH according to the formula

R—X—CO—OAr

wherein ArOH is a pharmaceutically active compound selected from the group consisting of SN-38, PI-103, etoposide and fenretinide, wherein

a) R is a residue of cholesterol, sitosterol, SN-38, PI-103, etoposide or fenretinide and X is O—CO-L, wherein L is either a direct bond or a linking group including a branched or unbranched hydrocarbyl moiety that may optionally include in-chain or pendant heteroatom substituents and/or cyclic moieties;

b) R—X—CO—O is an all-trans retinoate radical or the 9-cis or 13-cis isomer thereof; or

c) R—X— is a branched or unbranched, saturated or unsaturated hydrocarbyl moiety comprising at least 5 carbon atoms and optionally including at least one in-chain or pendant heteroatom substituent and/or cyclic moiety.

In another aspect, the invention provides nanoparticles of the ester described immediately above.

In yet another aspect, the invention provides a dispersion of nanoparticles in an aqueous medium. The nanoparticles, which are typically solid, include an ester of ArOH according to the formula

R—X—CO—OAr

wherein ArOH is a pharmaceutically active compound in which Ar is a substituted or unsubstituted aryl or heteroaryl radical, and wherein

a) R is a residue of tocopherol, cholesterol, sitosterol, SN-38, PI-103, etoposide or fenretinide and X is O—CO-L, wherein L is either a direct bond or a linking group including a branched or unbranched hydrocarbyl moiety that may optionally include in-chain or pendant heteroatom substituents and/or cyclic moieties;

b) R—X—CO—O is an all-trans retinoate radical or the 9-cis or 13-cis isomer thereof; or

c) R—X— is a branched or unbranched, saturated or unsaturated hydrocarbyl moiety comprising at least 5 carbon atoms and optionally including at least one in-chain or pendant heteroatom substituent and/or cyclic moiety.

The invention also provides a method of treating a diagnosed medical condition in a patient. The method includes administering to the patient one or more dosages of the ester, nanoparticles containing the ester, or the dispersion of nanoparticles as described above, wherein the one or more dosages constitute an amount therapeutically effective to treat the medical condition.

### BRIEF DESCRIPTION OF THE DRAWINGS

FIG. 1 shows the size distribution of PEGylated biodegradable nanoparticles formulated with the codrug conjugate of SN-38 and tocopherol succinate according to the invention.

FIG. 2A shows the therapeutic efficacy of biodegradable nanoparticles formulated with tocopherol succinate-SN38 codrug according to the invention, compared with orally administered irinotecan and a ‘no treatment’ control in the mouse xenograft model of neuroblastoma.

FIG. 2B shows animal survival data resulting from the experiments whose efficacy results are shown in FIG. 2A.

FIG. 3 shows results of tumor growth inhibition by PLA-PEG nanoparticles loaded with an SN-38 conjugate with tocopherol succinate, according to the invention.

FIG. 4 shows growth inhibition of large-sized tumors by NP loaded with an SN-38 conjugate with tocopherol succinate, according to the invention.

FIG. 5 shows the particle size distribution in a nanosuspension of an SN-38 conjugate with all-trans retinoic acid, according to the invention.

FIG. 6 shows growth inhibition of large-sized tumors by a human serum albumin-stabilized nanosuspension of SN-38 conjugated with all-trans retinoic acid, according to the invention.

### DETAILED DESCRIPTION OF THE INVENTION

The inventors have found that biodegradable nanoparticles offer an advantageous alternative way of delivering poorly water-soluble compounds, including for example pharmaceuticals, such as SN-38. Providing a pharmaceutical in this form can address the solubility issues and improve the pattern of drug biodistribution via passive or active targeting mechanisms favoring accumulation in the tumor, thereby effecting an improved therapeutic profile with reduced toxicity and/or increased efficacy.

However, the inventors have found that formulating drugs in nanoparticles capable of taking advantage of the EPR effect is challenging for at least two reasons. First, the few methods traditionally used for making sufficiently small, biodegradable polymer-based particles typically use chemical energy derived from redistribution of fully or partially water-miscible solvents into an aqueous medium (e.g., nanoprecipitation, emulsification-solvent diffusion). Although formulation processes governed by solvent diffusion are effective in forming small nanoparticles with narrow size distribution, the underlying mechanism is also the primary cause of the low drug entrapment yields typically observed with these methods, as the drug tends to migrate with the solvent into the continuous phase escaping the proto-nanoparticles. Second, the small size of the particles results in a high surface area to volume ratio, which accelerates the release of the particle-associated drug into the surrounding medium. This is undesirable.

The inventors have now found that these problems may be addressed by derivatizing the drug molecule to minimize its aqueous solubility and endow it with a higher affinity to a matrix material that may optionally be included in the particle. Thus, as opposed to previously known modifications aimed at increasing the hydrophilicity of drugs administered in a solubilized form, the invention provides a strategy for drug modifications aimed at reducing hydrophilicity rather than increasing it in order to enable effective encapsulation and delivery of the drug in the form of a nanoparticle. At the same time, upon release from the nanoparticle, the derivative is capable of rapidly regenerating the active parent drug without significant loss of pharmacological activity. The derivatized drug may be either a prodrug or a codrug, depending on whether it contains one or several drug precursor moieties (i.e., pharmacophores) within the molecule, respectively.

The nanoparticles may be in the form of a nanosuspension; i.e., a suspension in an aqueous medium where the suspended nanoparticles are colloidally stabilized, for example via an ionic or steric stabilizer (e.g., albumin), but in which the nanoparticles do not include a water-insoluble matrix material in addition to the drug substance. Alternatively, the nanoparticles may additionally include a water-insoluble matrix material, provided that it is biodegradable or bioeliminable. Nonlimiting examples include aliphatic polyesters (e.g., polylactides and copolymers thereof) and aliphatic polyanhydrides. Exemplary matrix materials include poly(D,L-lactide), poly(D,L-lactide)-poly(ethylene glycol) block copolymer, poly(L-lactide), poly(L-lactide)-poly(ethylene glycol) block copolymer, poly(epsilon-caprolactone), poly(epsilon-caprolactone)-poly(ethylene glycol) block copolymer, poly(lactide-co-glycolide), and poly(lactide-co-glycolide)-poly(ethylene glycol) block copolymer.

The invention provides highly lipophilic prodrug or codrug derivatives of select anticancer agents designed to provide improved incorporation into small-sized, injectable nanoparticles. The nanoparticles typically have an average diameter less than 200 nm, or less than 150 nm, or less than 100 nm, or less than 75 nm. Typically, the average diameter is at least 10 nm, or at least 20 nm, or at least 40 nm. The rapid recovery of the parent drugs from the prodrug or codrug conjugates upon release from the particles is governed by hydrolytic cleavage of phenolic ester bonds. The derivatization of SN-38 is detailed as a representative example of an anticancer agent amenable to modification via phenolic ester bridges to provide lipophilic prodrugs and codrugs that can be rapidly activated by hydrolysis to form the parent drug. The design and synthesis of such derivatives for SN-38 can be extended to most pharmaceutical compounds bearing a phenolic hydroxyl group, and all such derivatives are contemplated by this invention. Nonlimiting examples exhibiting different pharmacological effects relevant to cancer therapy, whose chemical structure enables phenolic ester derivatization, include 3-[4-(4-morpholinyl)pyrido[3′,2′:4,5]furo[3,2-d]pyrimidin-2-yl]-phenol (also referred to as PI-103), 4′-demethyl-epipodophyllotoxin 9-[4,6-O—(R)-ethylidene-beta-D-glucopyranoside] (known as etoposide) and N-(4-hydroxyphenyl)retinamide (also known as fenretinide or 4-HPR). The structures of these compounds are shown in Schemes 1-4 in the Examples, and examples illustrating their derivatization are provided (See examples 1-6).

The succinate ester of tocopherol (also referred to as tocopherol succinate or tocopherol hemisuccinate) was chosen as a complementary pharmacophore for some codrug derivatives due to its well-established anticancer efficiency (See Examples 1 and 2 below). All-trans retinoic acid and its 9-cis and 13-cis isomers, which have also been used successfully for treating different types of cancer, are also useful complementary pharmacophores for making highly lipophilic phenolic ester-based codrug derivatives of the abovementioned and other phenolic hydroxyl-containing compounds.

Suitable prodrugs can be prepared using hydrocarbyl moieties capable of providing sufficient lipophilicity, or sufficient hydrophobicity, to minimize water solubility. For example, one suitable prodrug can be prepared using succinyl cholesterol, which provides lipophilicity but is not biologically convertible into a pharmacologically active compound. Therefore, it was chosen as the promoiety for constructing the SN-38 prodrug in Example 3 below. Other suitable prodrugs can be prepared using other sufficiently lipophilic hydrocarbyl moieties.

In general, suitable prodrugs and codrugs include esters of ArOH according to the formula

R—X—CO—OAr

wherein ArOH is a pharmaceutically active compound in which Ar is a substituted or unsubstituted aryl or heteroaryl radical, and wherein

a) R is a residue of tocopherol, cholesterol, sitosterol, SN-38, PI-103, etoposide or fenretinide and X is O—CO-L, wherein L is either a direct bond or a linking group comprising a branched or unbranched hydrocarbyl moiety that may optionally comprise in-chain or pendant heteroatom substituents and/or cyclic moieties;

b) R—X—CO—O is an all-trans retinoate radical or the 9-cis or 13-cis isomer thereof; or

c) R—X— is a branched or unbranched, saturated or unsaturated hydrocarbyl moiety comprising at least 5 carbon atoms and optionally including at least one in-chain or pendant heteroatom substituent and/or cyclic moiety.

Typically, the hydrocarbyl moiety in the linking group L will comprise from 1 to 30 carbon atoms, or from 1 to 20, or from 1 to 10, or from 1 to 6. In some embodiments, Ar is phenyl bearing one or more substituents in addition to the OH moiety that forms the ester. Exemplary compounds ArOH include SN-38, PI-103, etoposide and fenretinide. In some embodiments, X is O—CO—(CH₂)₂ or O—CO—(CH₂)₂—CO—O—CH₂, Exemplary methods of making the prodrugs and codrugs are shown in the Examples. Other methods include those described in International Patent Application No. PCT/US11/67531, filed on 28 Dec. 2011, and in U.S. Provisional Patent Application No. 61/427,615, filed on 28 Dec. 2010, the entireties of which applications are incorporated herein by reference.

In some embodiments, R—X— may be chosen from branched or unbranched, saturated or unsaturated hydrocarbyl moieties comprising at least 5 carbon atoms, such as from 5 to 40 carbon atoms, or from 5 to 30 carbon atoms, or from 10 to 20 carbon atoms. The hydrocarbyl moiety may optionally comprise at least one in-chain or pendant heteroatom substituent and/or a cyclic moiety.

In some embodiments, R—X— is selected such that R—X—CO—O comprises a radical derived from carboxylic acids. For example, R—X—CO—O may comprise a radical derived from carboxylic acid comprising at least 5 carbon atoms, such as 5 to 40 carbon atoms, or from 5 to 30 carbon atoms, or from 10 to 10 carbon atoms. The carboxylic acid may be branched or unbranched, saturated or unsaturated, and may comprise at least one in-chain or pendant heteroatom or cyclic substituent. For example, the radical derived from a carboxylic acid may be derived from a branched carboxylic acid comprising a cyclic substituent, such as retinoic acid, or an unbranched carboxylic acid comprising a cyclic substituent comprising heteroatom substitutions, such as residue derived from a boron-dipyrromethene (BODIPY) based residue. Other carboxylic acids may comprise heteroatom substituents.

The radical derived from carboxylic acids may also comprise radicals derived from fatty acids. The fatty acids may comprise an aliphatic chain having 5 to 30 carbon atoms, or from 10 to 20 carbon atoms. The fatty acids may be saturated or unsaturated. Non-limiting examples of fatty acids that may be used include oleic acid, elaidic acid, docosahexaenoic acid, and eicosahexaenoic acid. Other biocompatible fatty acids, including omega-3, omega-6, and omega-9 fatty acids may also be used.

In some embodiments, R—X— may be chosen such that R—X—CO—O is a diester radical. The prodrugs or codrugs of the invention, and nanoparticles or dispersions thereof, may be used to treat a diagnosed medical condition in a patient. Such methods of treatment involve administering to the patient one or more dosages of the ester or nanoparticle or dispersion such that the one or more dosages constitute an amount therapeutically effective to treat the medical condition.

### Examples

The 10-hydroxy group of SN-38 was esterified with the corresponding free carboxylic acids by a standard method employing 1-ethyl-3-(3-dimethylaminopropyl)carbodiimide hydrochloride (EDC) as an activator and 4-dimethylaminopyridine tosylate (DPTS) as a catalyst at 0-25° C. in a mixture of 1-methylpyrrolidinone (1-MP) or N,N-dimethylacetamide (DMAc) and dichloromethane, yielding the lipophilic pro-drug conjugates (1a-c) as shown in Scheme 1.

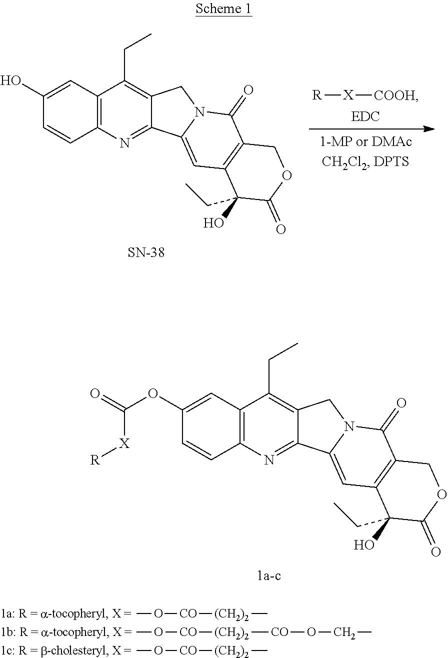

### Example 1

### Preparation of the Conjugate (1a)

SN-38 (240 mg, 0.60 mmol) and α-tocopheryl hemisuccinate (372 mg, 0.70 mmol) in a mixture of 1-MP (7.8 mL) and dichloromethane (3 mL) were sonicated for 5 min, and the resulting thin suspension was cooled in an ice-water bath. DPTS catalyst (110 mg, 0.37 mmol) and EDC (132 mg, 0.69 mmol) were added, the mixture was stirred in the bath for 10 min, warmed to room temperature (becoming homogeneous) and further stirred for 2 h. Another portion of EDC (204 mg, 1.06 mmol) was added, the stirring was continued for additional 15 h. Aqueous 5% solution of sodium dihydrophosphate (80 mL, acidified with phosphoric acid to pH=3) was added, the mixture was extracted with ethyl acetate (50 mL), the organic phase was washed with water (3×20 mL), with 5M aqueous sodium chloride (80 mL), and dried. The crude product was purified by flash chromatography (silica-gel, chloroform-ethyl acetate, 100:0 to 3:2). Yield of 1a: 423 mg (80%), the structure and purity were confirmed by TLC and 1H NMR.

### Example 2

### Preparation of the Conjugate (1b)

3-(α-tocopheryloxycarbonyl)propionyloxyacetic acid is first prepared as follows. α-Tocopheryl hemisuccinate (Sigma, ≧0.98%, 208 mg, 0.384 mmol) is neutralized with an equimolar amount of aqueous 40% tetrabutylammonium hydroxide. The resulting Bu₄N-salt is dried by co-evaporations in vacuo with 2-propanol and heptane, cooled to 0° C. dissolved in 1-methylpyrrolidinone (1.3 mL), and protected with the argon atmosphere. tert-Butyl bromoacetate (0.071 mL, 0.47 mmol) is added, the mixture is stirred at 0° C. for 1 h and diluted with water (15 mL). The separated ester is extracted with hexane (30 mL), and washed with water (2×15 mL). The combined aqueous layer is extracted with ethyl acetate (15 mL), the organic phase is washed with water (3×15 mL), and the combined extracts are dried. The crude ester is purified by flash chromatography (silica-gel, hexane-ethyl acetate, 100:0 to 10:1) The resulting tert-butyl ester (249 mg) is dissolved in dry CH₂Cl₂ (1.1 mL), protected with argon, and trifluoroacetic acid (0.70 mL) followed by triethylsilane (0.35 mL) is added. The mixture is left at 23° C. for 1.2 h, and dried in vacuo. The residue is purified by flash chromatography on silica-gel (CHCl₃-MeCN, 100:0 to 3:1) to yield 3-(α-tocopheryloxycarbonyl)propionyloxyacetic acid (194 mg) in 84% yield.

3-(α-tocopheryloxycarbonyl)propionyloxyacetic acid (89 mg, 0.151 mmol) and SN-38 (58 mg, 0.145 mmol) and in 1-MP (1.7 mL) and dichloromethane (0.7 mL) were sonicated, reacted with DPTS (50 mg, 0.170 mmol) followed by EDC (27 mg, 0.141 mmol then 55 mg, 0.287 mmol) and worked up as described in Example 1. The crude product was purified by flash chromatography (silica-gel deactivated with acetic acid, CHCl3-ethyl acetate, 100:0 to 3:2). Yield of 1b: 40 mg (29%), the structure and purity were confirmed by TLC and 1H NMR.

### Example 3

### Preparation of the Conjugate (1c)

A mixture of SN-38 (20 mg, 0.050 mmol), β-cholesteryl hemisuccinate (27 mg, 0.055 mmol) and DPTS catalyst (26 mg, 0.088 mmol) in DMAc (0.65 mL) and dichloromethane (0.2 mL) was stirred at room temperature, and EDC (11 mg, 0.056 mmol) was added. The stirring was continued for 2.5 h, and the second portion of EDC (22.mg, 0.11 mmol) followed by dichloromethane (1 mL) were introduced. The stirring at room temperature was continued for 8 h. The resulting thick suspension was diluted with aqueous 5% solution of sodium dihydrophosphate (20 mL, acidified with phosphoric acid to pH=3), extracted with ethyl acetate (25 mL), the organic phase was washed with water (3×10 mL), with 5M aqueous sodium chloride (3×25 mL), and dried. The crude product was purified by flash chromatography (silica-gel, chloroform-ethyl acetate, 100:0 to 3:2). Yield of 1c: 30 mg (70%), the structure and purity were confirmed by TLC and 1H NMR.

### Example 4

An analogous conjugate (3) of 3-(4-(4-morpholinyl)pyrido[3′,2′:4,5]furo[3,2-d]pyrimidin-2-yl)phenol (PI-103) and α-tocopheryl hemisuccinate was prepared as shown in Scheme 2. Analogous compounds in which R and X may be any of the combinations shown in Scheme 1 may also be prepared by similar reactions.

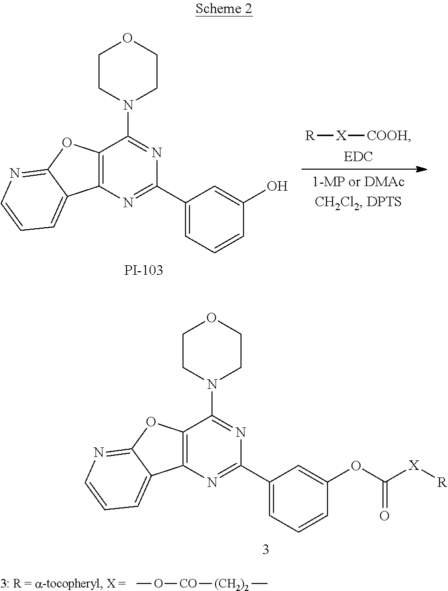

PI-103 (20 mg, 0.057 mmol) and α-tocopheryl hemisuccinate (40 mg, 0.075 mmol) DPTS catalyst (15 mg, 0.051 mmol) and EDC (24 mg, 0.125 mmol) were stirred in a mixture of 1-MP (1.0 mL) and dichloromethane (1.0 mL) for 19 h at room temperature. An aqueous 2.5M NaCl solution (20 mL) was added, the mixture was extracted with ethyl acetate (20 mL), the organic phase was washed with 2.5M NaCl (20 mL), with 10% KHCO₃ (20 mL), with water (10 mL), and dried. The crude product was purified by flash chromatography (silica-gel, chloroform-ethyl acetate, 100:0 to 4:1). Yield of 3: 47 mg (95%), the structure and purity were confirmed by TLC and ¹H NMR.

### Example 5

The synthesis of fenretinide hemisuccinate, which can be used to form part of prodrug and codrug constructs, is shown below in Scheme 3, along with a synthetic route for preparing such a codrug with SN-38.

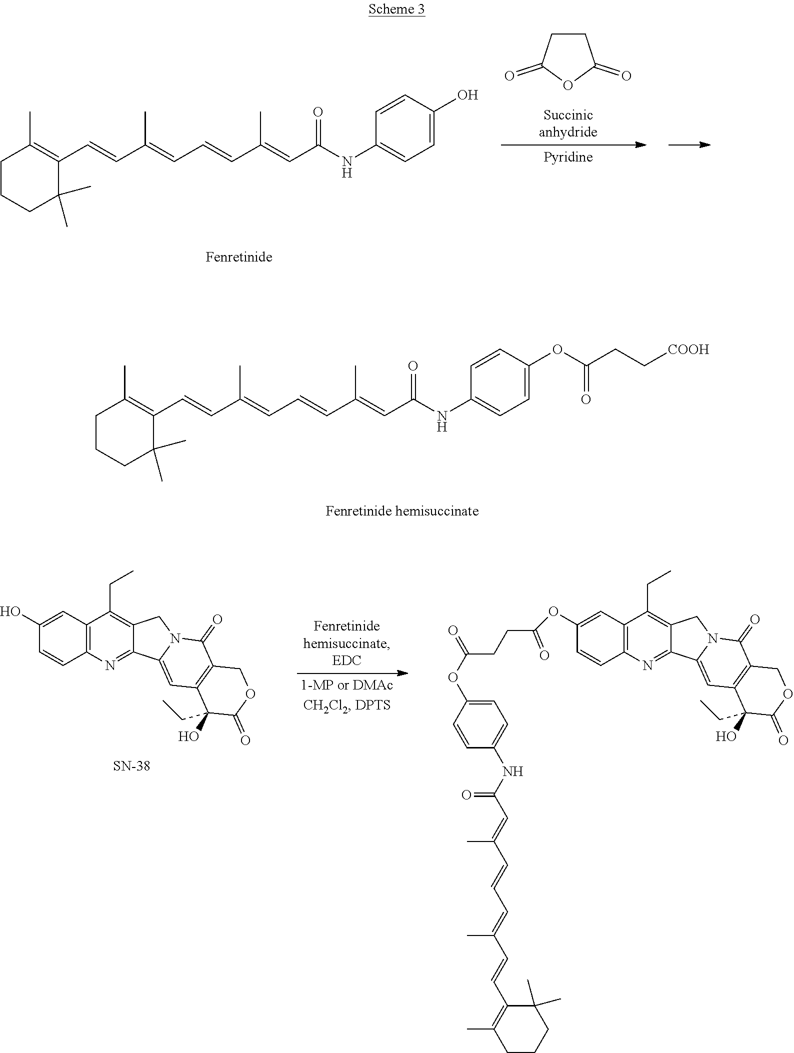 [SMILES: O=C1CCC(=O)O1 ; CC1=C(/C=C/C(C)=C/C=C/C(C)=C/C(=O)Nc2ccc(O)cc2)C(C)(C)CCC1 ; CC1=C(/C=C/C(C)=C/C=C/C(C)=C/C(=O)Nc2ccc(OC(=O)CCC(=O)O)cc2)C(C)(C)CCC1 ; CCc1c2c(nc3ccc(O)cc13)-c1cc3c(c(=O)n1C2)COC(=O)[C@]3(O)CC ; "SN-38" ; CCc1c2c(nc3ccc(OC(=O)CCC(=O)Oc4ccc(NC(=O)/C=C(C)/C=C/C=C(C)/C=C/C5=C(C)CCCC5(C)C)cc4)cc13)-c1cc3c(c(=O)n1C2)COC(=O)[C@]3(O)CC]

Fenretinide ((196 mg, 0.50 mmol) succinic anhydride (256 mg, 2.56 mmol), dichloromethane (1.35 mL) and pyridine (0.75 mL, 9.27 mmol) were stirred at room temperature for 66 h, protected with the argon atmosphere. An aqueous 1M solution of H₃PO₄ (65 mL) was added, the mixture was extracted with ethyl acetate (120 mL), the organic phase was washed with 1M H₃PO₄ (60 mL), with water (3×50 mL), filtered, and concentrated in vacuo to 2 mL. The separated solid was filtered off, washed with ethyl acetate (4×0.5 mL) and with pentane. Yield of fenretinide hemisuccinate: 227 mg (92%), the product was further purified by dissolving in a large amount of ethyl acetate and concentrating to a small volume. The structure and purity of fenretinide hemisuccinate were confirmed by TLC and ¹H NMR.

### Example 6

### Preparation of Conjugate from Etoposide and Tocopherol Hemisuccinate (2, Scheme 4)

Etoposide (40 mg, 0.067 mmol), α-tocopheryl hemisuccinate (42 mg, 0.079 mmol) and DPTS catalyst (15 mg, 0.051 mmol) in a mixture of 1-MP (0.9 mL) and dichloromethane (0.35 mL) were cooled in an ice-water bath and EDC (15 mg, 0.078 mmol) were added, the mixture (homogeneous) was stirred in the bath for 10 min, warmed to room temperature and further stirred for 2 h. Another portion of EDC (23 mg, 0.12 mmol) was added, the stirring was continued for additional 28 h. Aqueous 5% solution of sodium dihydrophosphate (20 mL, acidified with phosphoric acid to pH=3) was added, the mixture was extracted with ethyl acetate (30 mL), the organic phase was washed with water (3×25 mL), and dried. The crude product was purified by flash chromatography (silica-gel, chloroform-ethyl acetate, 100:0 to 1:1). Yield of 2: 39 mg (52%), the structure and purity were confirmed by TLC and ¹H NMR. Analogous compounds in which R and X may be any of the combinations shown in Scheme 1 may also be prepared by similar reactions.

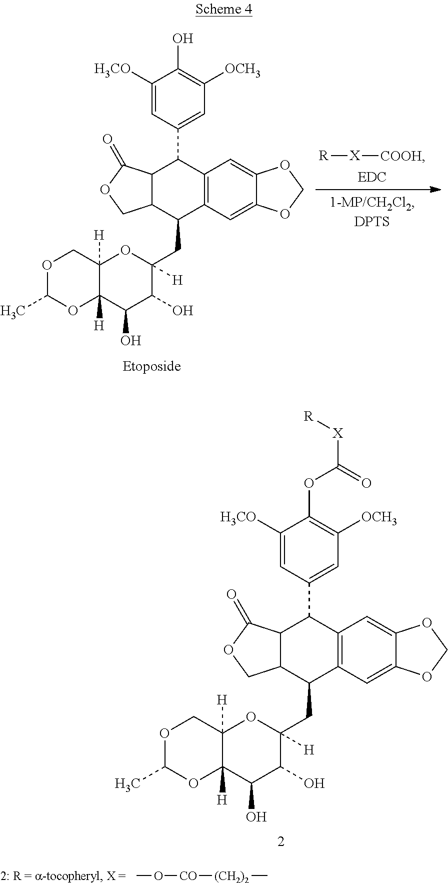

### Example 7

### Formulation of Small Sized Nanoparticles with the Codrug Conjugate of SN-38 and Tocopherol Succinate

PEGylated biodegradable nanoparticles were prepared using a modification of the nanoprecipitation method optimized for producing ultrasmall particulates. A 10 mg portion of SN-38 conjugate with tocopherol succinate synthesized as described in Example 1 and 20 mg of PLURONIC® F-68 surfactant were dissolved with sonication in 12 mL of acetone. A 200 mg portion of poly(D,L-lactide)-poly(ethylene glycol) block copolymer (50 kDa:5 kDa) was dissolved in the acetonic solution, and 8 mL of ethanol was added to the organic phase. The organic phase was rapidly added to 50 mL water with magnetic stirring. The mixture was transferred into an evaporation flask, and the solvents were removed by gradually reducing the pressure from 130 mbar to 40 mbar at 30° C. The formulation was additionally concentrated, glucose was added to the nanoparticle suspension at 5% w/v to adjust the tonicity, and the volume was adjusted to 5.0 mL. The resulting nanoparticles were sterilized by passing them through a 0.22 μm filter unit.

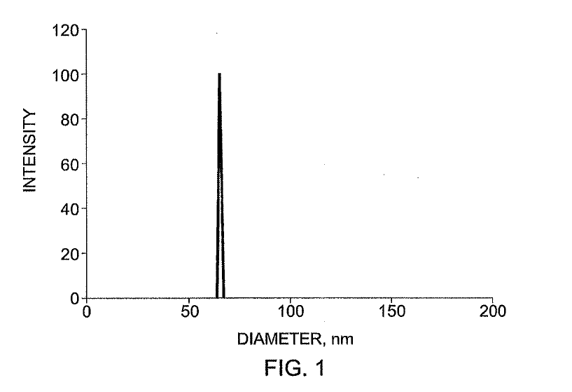

The drug was assayed spectrophotometrically against a suitable calibration curve after extraction in 2-butanol. The drug concentration in the nanoparticle formulation was 1.67 mg/mL, corresponding to an 84% incorporation yield. The particle size distribution was determined by dynamic light scattering, and is shown in FIG. 1.

### Example 8

### Tumor Growth Inhibitory Effect of Tocopherol Succinate-SN38 Codrug Formulated in Biodegradable Nanoparticles in the Murine Xenograft Model of Neuroblastoma

The SN-38 codrug conjugate with tocopherol succinate was synthesized as described in Example 1 and formulated in PEGylated biodegradable nanoparticles as described in Example 7.

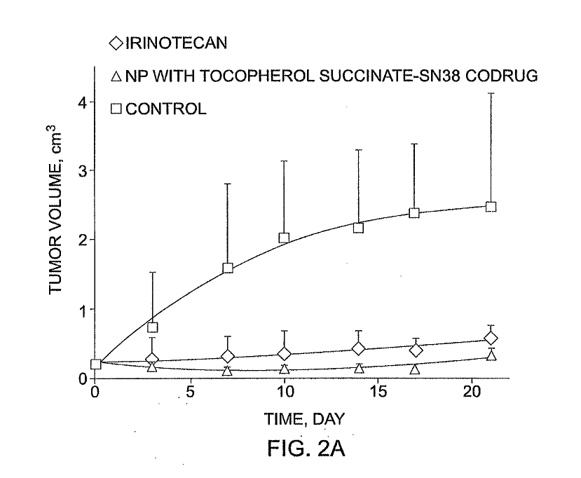

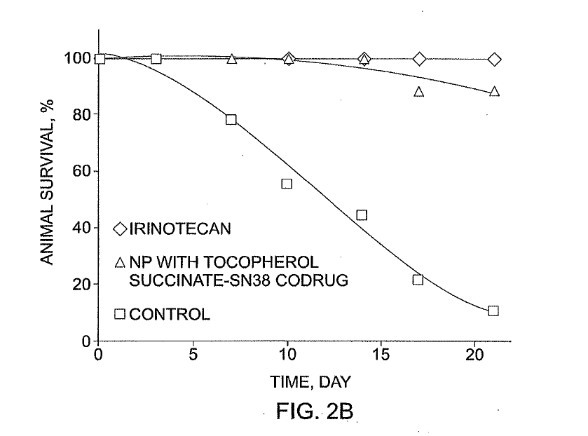

For xenograft studies, athymic nu/nu mice (nine animals per group) were injected in the flank with 10⁷ SY5Y-TrkB human neuroblastoma cells in 0.3 mL MATRIGEL™ matrix (BD Biosciences, Franklin Lakes, N.J.). Treatment was started when the average SY5Y-TrkB tumor size was 0.2 cm³. Nanoparticles were administered intravenously through the tail vein twice a week at a dose equivalent to 10 mg/kg of SN-38 per injection for comparison against a ‘no treatment’ control and a clinically used irinotecan formulation (CAMPTOSAR®, Pfizer, given orally five times a week at a therapeutically relevant dose of 10 mg/kg as described in Thompson J. et al., Efficacy of oral irinotecan against neuroblastoma xenografts, Anticancer Drugs. 1997 April; 8(4):313-22). Tumor size in each group was measured daily over a period of three weeks. While both nanoparticles and irinotecan showed efficacy vs. ‘no treatment’ control, the codrug-loaded nanoparticles administered twice a week (i.e., 20 mg/kg SN-38 per week) effectively inhibited tumor growth over the entire duration of the experiment and, in contrast to irinotecan administered at a considerably larger weekly dose (50 mg/kg drug per week), also notably reduced tumor size in the first 17 days (FIG. 2A). The error bars in FIG. 2A represent standard deviations. Both treatments resulted in significantly improved animal survival compared with the control group, where only one animal remained by the end of week 3 (FIG. 2B).

### Example 9

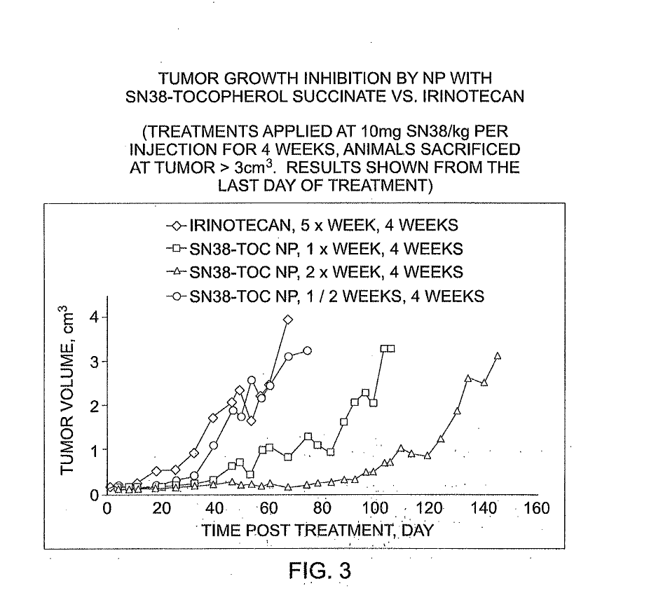

Tumor growth inhibition by PLA-PEG nanoparticles loaded with an SN-38 conjugate with tocopherol succinate (made according to Example 7) was administered over 4 weeks at indicated dose regimens (each injection equivalent to 10 mg SN-38/kg) to animals xenografted as in Example 8. The effect is shown in comparison to irinotecan administered 5 times a week for 4 weeks at the same dose per injection. Animals were sacrificed after their tumors reached 3 cm³. The results are shown in FIG. 3 as average tumor volumes starting from the last day of treatment.

Note that NP loaded with the SN-38 conjugate and administered once in two weeks (a total of 2 injections) were effective comparably with irinotecan given 5 times a week, while the other two regimens with more frequent dosing of NP (i.e. once and twice a week) were correspondingly more effective in inhibiting tumor growth and extending the animal survival.

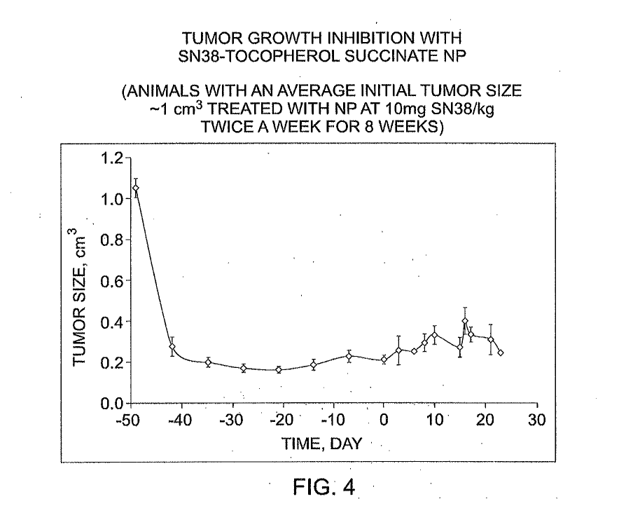

Growth inhibition of large-sized tumors by NP loaded with the SN-38 conjugate with tocopherol succinate is depicted in FIG. 4. NP administered twice a week caused rapid shrinkage of large-sized tumors from the initial size of ˜1 cm³ to 0.2 cm³. The formulation effectively prevented tumor regrowth during the entire treatment period (days −49 to 0) and for >3 weeks after the treatment was discontinued.

### Example 10

### Formulation of Nanosuspension of an SN-38 Conjugate with all-Trans Retinoic Acid

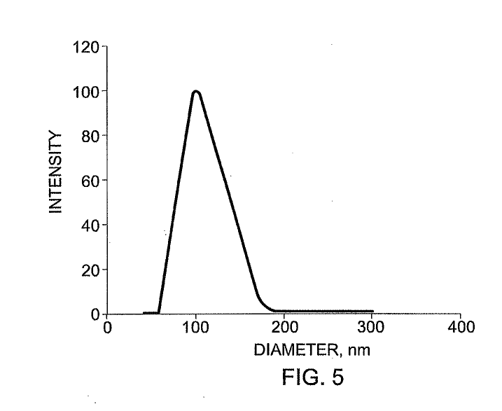

Ten mg SN-38 retinoate were dissolved in 2 ml acetone with a brief warming to 30° C. in a water bath, and the organic phase volume was adjusted with 10 ml acetone and 8 ml ethanol. The organic phase was rapidly added to 50 ml of an aqueous solution containing 200 mg human serum albumin with magnetic stirring. The mixture was transferred into an evaporation flask, and the solvents were removed by gradually reducing the pressure from 130 mbar to 40 mbar at 30° C. The formulation was additionally concentrated, trehalose was added to the nanosuspension at 10% w/v, and is the volume was adjusted to 4.0 ml. The obtained nanosuspension was sterilized by passing it through a 0.22 μm filter unit, frozen in 0.4-ml aliquots at −80° C., and lyophilized. The average particle size was 99 nm, determined by dynamic light scattering, and had the particle size distribution shown in FIG. 5.

### Example 11

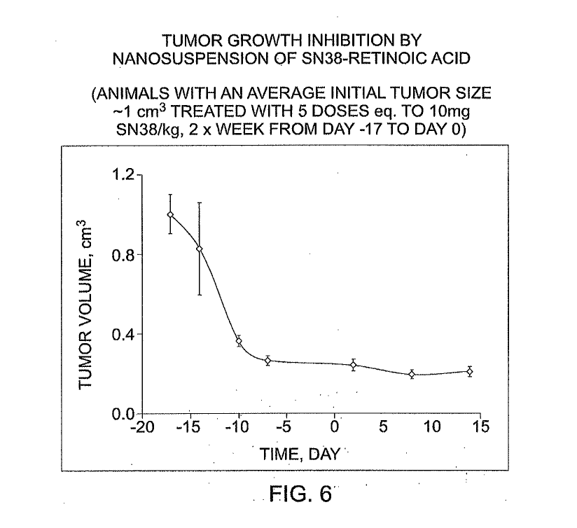

Growth inhibition of large-sized tumors by a human serum albumin-stabilized nanosuspension of SN-38 conjugated with all-trans retinoic acid, made according to Example 10, was determined. The treatment was administered twice a week at 10 mg SN-38/kg per injection, a total of 5 doses, with the results seen in FIG. 6. Note the rapid tumor shrinkage and effective prevention of tumor regrowth during and after the treatment period achieved with this formulation.

The present invention enables effective inclusion of potent, yet toxic drugs in the form of long-circulating nanoparticles of prodrugs or codrugs to achieve tumor targeting. The nanoparticles are of sufficiently small size for effective targeted delivery to tumors, and their use can ameliorate the otherwise significant adverse effects seen when the parent drug is administered in a solubilized form. The nanoparticles of this invention may improve the therapeutic indices of the parent drugs, and provide clinically and commercially viable modalities for treating neuroblastoma and other types of malignancies.

Although the invention is illustrated and described herein with reference to specific embodiments, the invention is not intended to be limited to the details shown. Rather, various modifications may be made in the details within the scope and range of equivalents of the claims without departing from the invention.

1. An ester of ArOH according to the formula
R—X—CO—OAr
wherein ArOH is a pharmaceutically active compound selected from the group consisting of SN-38, PI-103, etoposide and fenretinide, wherein
a) R is a residue of cholesterol, sitosterol, SN-38, PI-103, etoposide or fenretinide and X is O—CO-L, wherein L is either a direct bond or a linking group comprising a branched or unbranched hydrocarbyl moiety that may optionally comprise in-chain or pendant heteroatom substituents and/or cyclic moieties;
b) R—X—CO—O is an all-trans retinoate radical or the 9-cis or 13-cis isomer thereof; or
c) R—X— is a branched or unbranched, saturated or unsaturated hydrocarbyl moiety comprising at least 5 carbon atoms and optionally including at least one in-chain or pendant heteroatom substituent and/or cyclic moiety.

2. The ester according to claim 1, wherein X is O—CO—(CH₂)₂ or O—CO—(CH₂)₂—CO—O—CH₂.

3. The ester according to claim 1, wherein R—X—CO—O is a radical derived from a fatty acid comprising an aliphatic chain having 5 to 30 carbon atoms.

4. The ester according to claim 3, wherein R—X—CO—O is a radical derived from a fatty acid comprising an aliphatic chain having 10 to 20 carbon atoms.

5. The ester according to claim 1, wherein R—X—CO—O is a radical derived from oleic acid, elaidic acid, docosahexaenoic acid, or eicosahexaenoic acid.

6. A nanoparticle comprising the ester according to claim 1.

7. The nanoparticle according to claim 6, wherein the nanoparticle further comprises a biodegradable or bioeliminable matrix material.

8. The nanoparticle according to claim 7, wherein the matrix material comprises a poly(D,L-lactide)-poly(ethylene glycol) block copolymer.

9. A dispersion of solid nanoparticles in an aqueous medium, wherein the nanoparticles comprise an ester of ArOH according to the formula
R—X—CO—OAr
wherein ArOH is a pharmaceutically active compound in which Ar is a substituted or unsubstituted aryl or heteroaryl radical, and wherein
a) R is a residue of tocopherol, cholesterol, sitosterol, SN-38, PI-103, etoposide or fenretinide and X is O—CO-L, wherein L is either a direct bond or a linking group comprising a branched or unbranched hydrocarbyl moiety that may optionally comprise in-chain or pendant heteroatom substituents and/or cyclic moieties;
b) R—X—CO—O is an all-trans retinoate radical or the 9-cis or 13-cis isomer thereof; or
c) R—X— is a branched or unbranched, saturated or unsaturated hydrocarbyl moiety comprising at least 5 carbon atoms and optionally including at least one in-chain or pendant heteroatom substituent and/or cyclic moiety.

10. The dispersion of nanoparticles according to claim 9, wherein Ar is phenyl bearing one or more substituents in addition to the OH moiety that forms the ester.

11. The dispersion of nanoparticles according to claim 9, wherein ArOH is selected from the group consisting of SN-38, PI-103, etoposide and fenretinide.

12. The dispersion of nanoparticles according to claim 9, wherein X is O—CO—(CH₂)₂ or O—CO—(CH₂)₂—CO—O—CH₂.

13. The dispersion of nanoparticles according to claim 9, wherein R—X—CO—O is a radical derived from a fatty acid comprising an aliphatic chain having 5 to 30 carbon atoms.

14. The dispersion of nanoparticles according to claim 9, wherein R—X—CO—O is a radical derived from a fatty acid comprising an aliphatic chain having 10 to 20 carbon atoms.

15. The dispersion of nanoparticles according to claim 9, wherein R—X—CO—O is a radical derived from oleic acid, elaidic acid, docosahexaenoic acid, or eicosahexaenoic acid.

16. The dispersion of nanoparticles according to claim 9, wherein the nanoparticles further comprise a biodegradable or bioeliminable matrix material.

17. The nanoparticle according to claim 16, wherein the matrix material comprises a poly(D,L-lactide)-poly(ethylene glycol) block copolymer.

18. The dispersion of nanoparticles according to claim 9, wherein the dispersion is a nanosuspension.

19. A method of treating a diagnosed medical condition in a patient, comprising administering to the patient one or more dosages of the ester according to claim 1, wherein the one or more dosages constitute an amount therapeutically effective to treat the medical condition.
